(19) United States
(12) Patent Application Publication
(10) Pub. No.: US 2015/0285825 A1
(43) Pub. Date: Oct. 8, 2015

(54) COMPOSITION OF CONJUGATES AND USES

(71) Applicant: REPROPHARM, Nouzilly (FR)

(72) Inventors: Jeremy Decourtye, Tours (FR); Elodie Kara, Tours (FR); Laurence Dupuy, Villedomer (FR); Marie-Christine Maurel, Tours (FR)

(21) Appl. No.: 14/421,485

(22) PCT Filed: May 6, 2013
(86) PCT No.: PCT/FR2013/051013

§ 371 Date: Jun. 15, 2015

(30) Foreign Application Priority Data
Aug. 13, 2012 (FR) 1257780

(51) Int. Cl. G01N 33/76; G01N 33/543

(52) U.S. Cl. CPC G01N 33/76; G01N 33/54393

(57) ABSTRACT

The present invention relates to a composition of conjugates and its use in a method of detecting compounds, for example biological molecules, for example peptides, proteins, hormones, nucleic acids, antibodies in a sample, for example a biological sample. The present invention relates in particular to a composition of conjugates and its use in a method of detecting the pre-ovulatory spike in LH in a biological sample originating from mammals and a kit for implementing the method of detection. The present invention finds an application in particular in the veterinary and medical fields.

### TECHNICAL FIELD

The present invention relates to a composition of conjugates and to the use thereof in a method for detecting compounds, for example biological molecules, for example peptides, proteins, protein, glycoprotein or steroid hormones, nucleic acids, antibodies, in a sample, for example a biological sample.

The present invention also relates to a composition of conjugates and the use thereof in a method for detecting the pre-ovulatory spike in LH in a biological sample originating from mammals and to a kit for implementing the detection method.

The present invention finds its application in particular in the medical and veterinary fields.

In the description below, the references between square brackets ([ ]) refer back to the list of references provided at the end of the text.

### PRIOR ART

The ovulation cycle in mammals involves a complex hormonal process involving in particular the pituitary gonadotrophic hormones: luteinizing hormone (LH) and follicle-stimulating hormone (FSH).

An ovulation-detecting test based on the detection of LH currently exists for female dogs and cats. However, this test is not 100% reliable and the results obtained sometimes comprise a significant number of false positives.

Ovulation tests based, for example, on an agglutination reaction or on an immunoenzymatic reaction also exist for female human beings. Many of these tests are provided in the form of a strip and are performed on urine, in which they detect the presence or absence of LH. However, the interpretation of the results obtained can be ambiguous, namely if there is the presence or absence of a second colored band, and creates a significant number of false positives. In this case, these methods lead to a poor interpretation and do not make it possible to accurately determine the moment at which ovulation will occur.

Moreover, these tests, which are all urine tests, are difficult to use for mammalian animals such as pigs, members of the ovine family, bovines or members of the goat family, or then require the presence of an individual for collecting the urine from the mammal, which in practice cannot be carried out and involves additional costs.

Two other types of test exist for women, one is based on measuring the pH of the skin via sweat using a microprocessor held in close contact with the skin by means of a scratch bracelet worn on the wrist. The other test is based on the observation, using a small microscope, of dried saliva which forms various patterns according to the level of estrogens secreted during a menstrual cycle. It requires carrying out the examination on perfectly clean saliva taken before eating, which is impossible with animals. These two expensive devices which require specific equipment are unsuitable for animals. Furthermore, the parameters measured are less precise than the pre-ovulatory spike in LH for reliably dating ovulation.

In addition, the methods known in the prior art for human beings are not directly applicable to other mammals, owing to the different specificities of the antibodies used and/or to the totally unacceptable cost that they entail.

There is therefore a real need to find a method and a test which overcome these deficiencies, drawbacks and obstacles of the prior art, in particular a method which makes it possible to detect the LH spike on any mammal with increased sensitivity. In addition, there is a real need to find a method which enables a simple interpretation of the result that is unambiguous and reliable over time and is inexpensive.

In particular, there is a real need to find a stable composition which makes it possible to store the reagents required for carrying out these tests, for example in a refrigerator, and inexpensively.

### DESCRIPTION OF THE INVENTION

The objective of the present invention is precisely to overcome the drawbacks of the prior art by providing a composition of conjugates comprising an antibody coupled to an enzyme, mammalian serum and glycerol.

In particular, a subject of the present invention is a composition of conjugates comprising an antibody coupled to an enzyme, at least two different mammalian sera, and glycerol.

According to the invention, an antibody coupled to an enzyme may be a polyclonal or monoclonal antibody. According to the invention, the antibody coupled to an enzyme may be a rabbit, ovine, caprine, equine, bovine, porcine, mouse or rat antibody.

In other words, according to the invention, the antibody coupled to an enzyme may be an antibody made in rabbits, members of the ovine family, members of the goat family, members of the horse family, bovines, pigs, mice or rats.

According to the invention, the antibody coupled to an enzyme may be an antibody directed against a molecule, for example a chemical molecule and/or a biological molecule, for example a protein, an amino acid sequence, a nucleic acid sequence, a glycoprotein hormone, for example luteinizing hormone, follicle-stimulating hormone, growth hormone, a steroid hormone, for example progestogens, for example progesterone, for example estrogens, for example estradiol, for example androgens, for example testosterone.

According to the invention, the concentration of antibody coupled to an enzyme may be from 2 to 50 μg/ml.

According to the invention, the antibody may be coupled to an enzyme selected from the group comprising the enzyme peroxidase, beta-galactosidase, glucose oxidase and alkaline phosphatase. Preferably, the enzyme is peroxidase, for example a heme peroxidase or a non-heme peroxidase. It may, for example, be a horseradish peroxidase sold by the company Sigma under the catalog reference P6782.

According to the invention, the term “different mammalian serum” is intended to mean sera originating respectively from different mammals, which may be of different species or of different ages or of different sexes.

According to the invention, the concentration of said at least two different mammalian sera may be included from 2% to 75% by volume, from 5% to 50% by volume, relative to the total volume of the composition of the present invention.

According to the invention, the term “mammalian serum” is intended to mean mammalian serum or normal mammalian serum.

According to the invention, the mammalian serum may be human or animal serum. It may, for example, be serum from a farm animal, from a pet or from another animal. For example, the farm animal may be selected from the group comprising bovines, pigs, members of the ovine family, members of the goat family, members of the camel family, canines, members of the horse family, murines and primates. For example, the pet may be selected from the group comprising canines and felines. The serum may for example be serum chosen from fetal calf serum, rabbit serum, or a mixture thereof.

According to the invention, the concentration by volume of mammalian serum may be included from 2% to 75% by volume, or from 5% to 50% by volume, relative to the total volume of the composition of the present invention.

According to the invention, the term “normal mammalian serum” is intended to mean serum obtained from a mammal which is healthy and has never been immunized against a particular antigen.

According to the invention, the normal mammalian serum may be human or animal serum. It may, for example, be normal serum from a farm animal, from a pet, or from any other animal, for example bovines, pigs, members of the ovine family, members of the goat family, members of the camel family, canines, members of the horse family, murines, felines and primates. It may, for example, be normal serum selected from normal fetal calf serum, normal rabbit serum, or a mixture thereof. In the present text, the normal rabbit serum may be commercially available serum; for example it may be the normal rabbit serum sold by the company Dutscher 2012 under the catalog reference P30-1101 or serum obtained from rabbits which have never been immunized against a particular antigen.

According to the invention, the concentration by volume of normal serum may be included from 2% to 60% by volume, from 4% to 50% by volume, relative to the total volume of the composition of the present invention.

In the present text, the glycerol may be commercially available glycerol; for example, it may be the glycerol sold by the company Sigma under the catalog reference G7893.

According to the invention, the composition of conjugates may also comprise from 1% to 30% by volume of glycerol relative to the total volume of the composition, from 1% to 20% by volume of glycerol relative to the total volume of the composition of the present invention.

According to the invention, the composition of conjugates may be in any form known to those skilled in the art. It may, for example, be an aqueous solution, an oil-in-water or water-in-oil emulsion, a multiple emulsion, a microemulsion, a solid emulsion, or an aqueous or aqueous-alcoholic gel.

Advantageously, the inventors have surprisingly shown that the composition of conjugates according to the invention can be stored at −20° C. and/or at 4° C. without complete alteration and/or loss of the biological/physicochemical properties of said composition and of the constituents thereof. In particular, the inventors have surprisingly shown that the composition according to the invention makes it possible to preserve the biological activity of the antibody coupled to an enzyme even if the composition is placed at a temperature of between −20° C. and 4° C.

A subject of the present invention is also the use of the composition of conjugates according to the invention in a method for detecting compounds, for example biological molecules/compounds, for example nucleic acids, antibodies, peptides, proteins, for example vascular endothelial growth factor (VEGF), endothelial growth factor (EGF), hormones, for example growth hormones, for example somatotropin, gonadotrophic hormones, for example luteinizing hormone (LH), follicle-stimulating hormone (FSH), steroid hormones, for example progestogens, for example progesterone, for example estrogens, for example estradiol, for example androgens, for example testosterone, in a sample, for example a biological sample.

In the present text, the term “method for detecting compounds” is intended to mean, for example, an immunoenzymatic method, for example an ELISA method, and/or any method for detecting compounds, for example biological, known to those skilled in the art.

According to the invention, the term “biological sample” is intended to mean a liquid or solid sample. According to the invention, the sample may be any biological fluid; for example, it may be a blood sample, plasma sample, serum sample, vaginal mucous sample, nasal mucous sample, saliva sample, urine and/or milk sample, tear sample or sweat sample. According to the invention, the sample may be a sample taken beforehand from said mammal.

According to the invention, the composition of conjugates of the invention may also be used in a method for detecting the pre-ovulatory spike in LH.

This may, for example, be a method for detecting the pre-ovulatory spike in LH in a biological sample originating from mammals and comprising the following steps: a) attaching an anti-LH antibody to a test surface; b) bringing the test surface on which said anti-LH antibody is attached into contact with a buffer solution comprising from 5% to 50% by volume of fetal calf serum; c) bringing said surface obtained in step (b) into contact with a biological sample; d) after step (c), rinsing the test surface in a washing solution; e) bringing the test surface rinsed in step (d) into contact with a buffer solution of conjugates comprising an anti-LH antibody coupled to an enzyme and from 5% to 50% by volume of fetal calf serum; f) after step (e), rinsing the test surface in a washing solution; and g) after step (f), bringing the test surface into contact with a solution comprising a substrate for said enzyme.

According to the invention, the buffer solution of conjugates may also comprise from 5% to 50% by volume, from 5% to 10% by volume, preferably 5% by volume, of normal rabbit serum.

Advantageously, the use of normal rabbit serum in the solution of conjugates makes it possible to increase the sensitivity of the method for detecting the spike in LH. According to the invention, the buffer solution of conjugates may also comprise from 1% to 30% by volume of glycerol, preferably from 1% to 15% by volume of glycerol.

Advantageously, the use of glycerol in the solution of conjugates makes it possible to increase the sensitivity of the method for detecting the spike in LH .

Advantageously, when the buffer solution of conjugates also comprises normal rabbit serum and glycerol, said solution can be stored at a temperature between 4° C. and −20° C. without deterioration of the properties of the antibody coupled to an enzyme.

According to the invention, the buffer solution of conjugates can be prepared prior to or concomitantly with the implementation of the method for detecting compounds, for example the abovementioned method.

A subject of the present invention is also the use of the composition of conjugates in a kit for implementing a method for detecting compounds, for example a method as described above.

Advantageously, the composition of conjugates according to the invention allows an increase in the sensitivity of the method for detecting compounds and of the kit in which it is used.

Other advantages will be able to further appear to those skilled in the art on reading the examples below, illustrated by the appended figures, given by way of illustration.

### BRIEF DESCRIPTION OF THE FIGURES

FIG. 1 represents a photograph of sticks after the implementation of the method of the invention in which the conjugate buffer optionally comprises 5% of normal rabbit serum and the AC2 HRP is at a concentration of 5 μg/ml. Samples of bovine plasmas comprising a concentration of 0 or 5.8 ng/ml of LH were used.

FIG. 2 represents a photograph of sticks after the implementation of the method of the invention in which the conjugate buffer comprises 5% by volume or 20% by volume of normal rabbit serum and the AC2 HRP is at a concentration of 5 μg/ml. The method was carried out with samples of bovine plasmas comprising a concentration of 0 or 5.8 ng/ml of LH.

FIG. 3 represents a photograph of sticks after the implementation of the method of the invention in which the conjugate buffer comprises 5, 10, 20, 25 or 50% by volume of normal rabbit serum and the AC2 HRP is at a concentration of 5 μg/ml. It was carried out with samples of bovine plasmas comprising a concentration of 0 ng/ml of LH.

FIG. 4 represents a photograph of sticks after the implementation of the method of the invention in which the conjugate buffer comprises 0.5% or 25% by volume of normal rabbit serum and the AC2 HRP is at a concentration of 10 μg/ml. The method was carried out with samples of bovine plasmas comprising a concentration of 0 or 5.8 ng/ml of LH.

FIG. 5 represents a photograph of sticks after the implementation of the method of the invention in which the conjugate buffer comprises 5% by volume of normal rabbit serum and 0, 1, 2, 3, 4, 5, 10, 20, 30, 40 or 50% by volume of glycerol and the AC2 HRP is at a concentration of 5 μg/ml. It was carried out with samples of bovine plasmas comprising a concentration of 0 or 7 ng/ml of LH.

FIG. 6 represents a photograph of sticks after the implementation of the method of the invention in which the conjugate buffer comprises 5% by volume of normal rabbit serum and 10, 15, 20, 25 or 30% by volume of glycerol and the AC2 HRP is at a concentration of 10 μg/ml. It was carried out with samples of bovine plasmas comprising a concentration of 0 or 7 ng/ml of LH.

FIG. 7 represents a photograph of sticks after the implementation of the method of the invention in which the conjugate buffer used was stored at −20° C. or 4° C. for 3 weeks and comprises in particular 5% by volume of normal rabbit serum and 1% by volume of glycerol and the AC2 HRP is at a concentration of 5 μg/ml. It was carried out with samples of bovine plasmas comprising a concentration of 0 or 6 ng/ml of LH.

FIG. 8 represents a photograph of sticks in which the conjugate buffer comprises 5% by volume of normal rabbit serum and 10% by volume of glycerol and the AC2 HRP is at a concentration of 10 μg/ml. It was carried out with samples of bovine plasmas comprising a concentration of 0 or 6 ng/ml of LH.

### EXAMPLES

### Example 1

### Method of Production and Composition of Conjugates Comprising Fetal Calf Serum, Normal Rabbit Serum and Glycerol

A composition comprising antibodies coupled to an enzyme (conjugates) was prepared as follows:

The method for preparing the anti-LH antibody coupled to horseradish peroxidase (HRP; supplier Sigma, reference P6782), referred to below as AC2 HRP, at 10 μg/ml, comprises the following steps:
1—an amount of AC2 HRP required to have a final concentration of 10 μg/ml was added and incubated in a volume of normal rabbit serum corresponding to 5% of the volume of the final preparation;
2—the mixture was then incubated for 45 minutes at 37° C.;
3—a required volume of PBS-FCS 50% (supplier Lonza, reference 14-801F) containing 0.05% by volume of ProClin300 (registered trademark, supplier Sigma-Aldrich, reference 48912-U) and 10% by volume of glycerol (supplier Sigma, reference G7893) was added in order to obtain a solution of AC2 HRP at a final concentration of 10 μg/ml;
4—the mixture obtained in step 3 was then incubated for 15 minutes at 37° C. and 300 μl of the incubated mixture were distributed per cryotube (Nunc cryotube, Dutscher, ref. 055003);
5—storage at 4° C. with the other constituents of the test.

In other words, for a final volume of 3 ml of a solution of AC2 HRP, i.e. an example of a composition of conjugates, at 10 μg/ml:
1—30 μg of AC2 HRP were incubated in 150 μl of rabbit serum;
2—incubation for 45 minutes at 37° C.;
3—addition of 2.85 ml of PBS-FCS 50% by volume containing 0.05% by volume of ProClin300, and 10% by volume of glycerol;
4—incubation for 15 minutes at 37° C. and distribution of 300 μl per cryotube;
5—storage at 4° C.

The normal rabbit serum used in step 1 was obtained by taking a blood sample from non-immunized rabbits: the term normal rabbit serum is used. It can also be purchased commercially (supplier Dutscher 2012 under the catalog reference P30-1101). The composition of the PBS was 0.01M K₂HPO₄ (supplier VWR; reference 26930.293), 0.01M KH₂PO₄ (supplier VWR; reference 26936.293), 0.15M NaCl (supplier VWR; reference 27810.295), pH 7.4. The PBS is prepared in milliQ water and filtered through a filter with a 0.22 μm porosity (supplier Millipore; reference GSWPO4700).

An anti-LH antibody coupled to peroxidase (AC2 HRP) was prepared at 5 μg/ml according to the method described above in 5% by volume of normal rabbit serum, and then diluted in PBS-FCS 50% by volume (supplier Lonza, reference 14-801F) containing 0.05% by volume of ProClin300 (registered trademark, supplier Sigma-Aldrich, reference 48912-U) and also comprising from 1% to 50% by volume of glycerol (supplier Sigma, reference G7893).

### Example 2

### Implementation of a Method for Detecting Molecules With a Solution of Conjugates (AC2 HRP) Optionally Comprising 5% of Normal Rabbit Serum

In Examples 2 to 9, the stick is the one sold by the company Nunc (Immuno™ Stick Nunc, Maxisorp), the ProClin300 (registered trademark) is sold by the company Sigma-Aldrich, ref. 48912-U, the fetal calf serum (FCS) came from the company Lonza, under the reference 14-801F, and the TMB membrane is the TMB membrane sold by the supplier KPL under the reference 50-77-18.

In this example, the detection method is a method for detecting the spike in LH. The test surface used was a plastic surface of a stick. An anti-LH antibody (AC1) was attached to the test surface by coating with 250 μl of rabbit anti-bovine LH antibody obtained, after purification on a protein A sepharose column, from a serum of a rabbit immunized with purified bovine LH, referred to below as AC1, prepared at 20 μg/ml in 0.1 M NaHCO₃/Na₂CO₃ buffer, pH 9.6, containing 0.05% by volume of ProClin300. The surface was then incubated for 1 hour at 37° C., then for 18 hours at 4° C. The surface was then drained by shaking in order to remove the rest of the AC1 liquid. The test surface to which the antibody was attached was brought into contact, via saturation coating of the surface, with 900 μl of PBS supplemented with 50% by volume fetal calf serum (FCS), for 1 hour at 37° C. Said surface obtained was then drained by shaking and dried vertically in an incubator at 37° C. for 3 hours.

The surface obtained was brought into contact with a sample of 250 μl of blood by immersion in a heparinized tube (Nunc polypropylene cryotube, ref. 368632). The heparinized tube was pretreated with 12 μl of a heparin solution at 500 IU/ml prepared from Heparin Choay sold by Sanofi Aventis and diluted in PBS filtered through a filter with a porosity of 0.2 μm (Millipore, ref. GSWPO4700), then dried for 3 hours in an incubator at 37° C. The test surface was then rinsed in 30 ml of a washing solution, namely a phosphate buffered saline solution PBS, pH 7.4, 0.01M K₂HPO₄/KH₂PO₄—0.15M NaCl comprised in a 40 ml tube. The rinsed test surface obtained was brought into contact, in a cryotube (sold by the company Nunc, ref. 368632), with 300 μl of a buffer solution of conjugate comprising a horse anti-ovine LH antibody coupled to horseradish peroxidase (HRP) prepared according to the method described in “Techniques Immuno-enzymatiques” [“Immunoenzymatic Techniques”] (Therese Ternynck and Stratis Avrameas, INSERM publications, 1987) referred to below as AC2 HRP, at 10 μg/ml in PBS-FCS 50% by volume containing 0.05% by volume of ProClin300 (registered trademark) previously prepared.

Under the conditions termed “without serum”, the AC2 HRP was prepared at 5 μg/ml in a solution of PBS-FCS 50% by volume containing 0.05% by volume of ProClin300.

Under the conditions termed “with 5% of rabbit serum”, the 5 μg/ml AC2 HRP was first incubated for 45 minutes at 37° C. in a volume of normal rabbit serum corresponding to 5% of the final volume of the AC2 HRP, then diluted in PBS-FCS 50% containing 0.05% by volume of ProClin300.

In each of these cases, two cow plasmas at 0 or 5.8 ng/ml of LH were used.

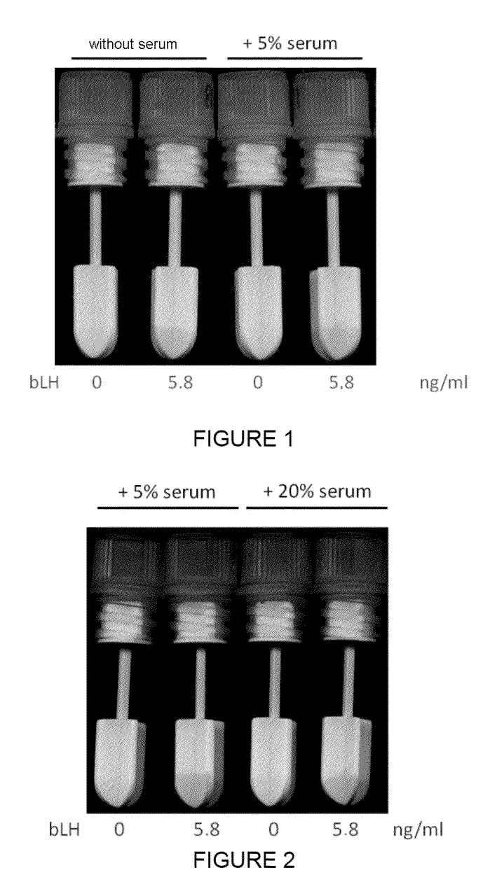

The test surface was then rinsed in 30 ml of a washing solution, namely a phosphate buffered saline solution PBS, pH 7.4, 0.01M K₂HPO₄/KH₂PO₄—0.15M NaCl comprised in a 40 ml tube. The rinsed test surface obtained was brought into contact for 15 minutes, in a cryotube sold by the company Nunc, ref. 368632, with a solution comprising a substrate for horseradish peroxidase (HRP), namely 300 μl of TMB membrane. The surface obtained was then removed from the tube and visual observation of the blue coloration on the test surface was carried out and showed the presence of LH in the sample tested. In this example, the quantification of the colored signal was carried out as described in the examples above, i.e. the sticks were scanned using an Epson scanner (Perfection 1200 Photo) and then the strength of the color obtained on each of them was quantified by densitometry with the “Scion Image” software (Scion Incorporation). This quantification is expressed in density units. FIG. 1 is a photograph of the sticks after revealing.

TABLE 1
Strength of the coloration obtained according to the presence
or absence of normal rabbit serum in the conjugate solution

| | |
|---|---|
| Without serum | +5% rabbit serum |

| LH (ng/ml) | 0 | 5.8 ng/ml | 0 | 5.8 ng/ml |
|---|---|---|---|---|
| Quantification of | 11.43 | 26.4 | 4.97 | 24.85 |
| the colored signal | | | | |

The method for preparing the AC2 HRP in 5% of normal rabbit serum makes it possible to reduce the background noise, without modifying the strength of the signal obtained with the plasma at 5.8 ng/ml.

In other words, the implementation of a method for detecting the spike in LH using a buffer solution of conjugate comprising an anti-LH antibody coupled to an enzyme, at 5 μg/ml, 50% of fetal calf serum and 5% of normal rabbit serum advantageously makes it possible to increase the sensitivity of detection by reducing the nonspecific signal.

### Example 3

### Comparative Tests of the Addition of 5% to 20% of Normal Rabbit Serum to the AC2 HRP Prepared at 5 μg/ml

The AC2 HRP was prepared at 5 μg/ml in 5% or 20% of normal rabbit serum, then diluted in PBS-FCS 50% by volume containing 0.05% by volume of ProClin300, in order to compare the effect of the normal rabbit serum on the background noise and the signal obtained after incubation of the stick in two cow plasmas at 0 and 5.8 ng/ml of LH, respectively. In this example, the quantification of the colored signal was carried out as described in Example 2 above. FIG. 2 is a photograph of the sticks after revealing.

TABLE 2
Strength of the coloration obtained according to the amount
of normal rabbit serum in the conjugate solution

| | |
|---|---|
| +5% serum | +20% serum |

| LH (ng/ml) | 0 | 5.8 ng/ml | 0 | 5.8 ng/ml |
|---|---|---|---|---|
| Quantification of | 10.28 | 26.38 | 10.28 | 24.26 |
| the colored signal | | | | |

The increase in the percentage of rabbit serum used to prepare the AC2 HRP does not modify the strength of the signal obtained on the stick. The preparation of the AC2 HRP in 5% or 20% of normal rabbit serum gives an equivalent result. In other words, the implementation of a method for detecting the spike in LH using one or other of the buffer solutions of conjugate does not modify the detection of the spike in LH.

### Example 4

### Comparative Tests of the Addition of 5% to 50% of Normal Rabbit Serum to the AC2 HRP Regarding the Nonspecific Signal of a Method for Detecting Compounds

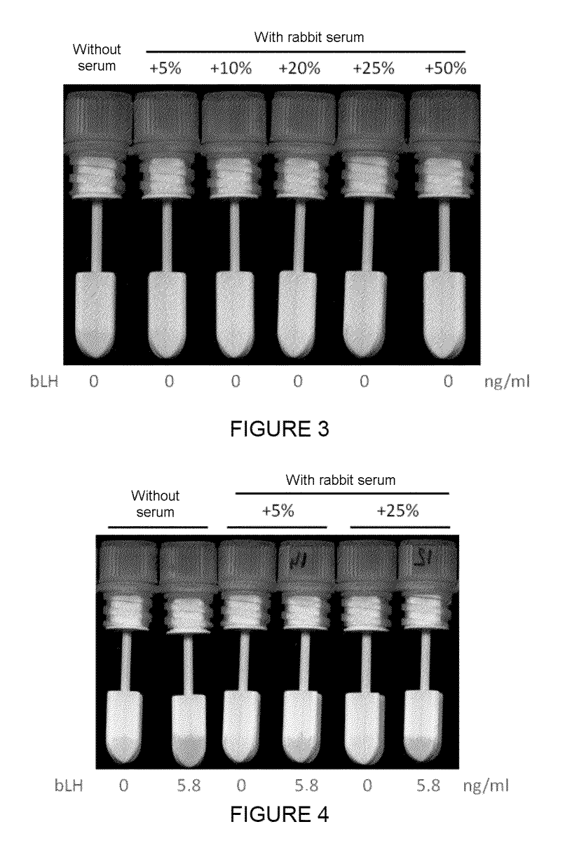

The AC2 HRP was prepared at 5 μg/ml according to the method described in Example 1 above, in 5%, 10%, 20%, 25% and 50% by volume of normal rabbit serum, then diluted in PBS-FCS 50% containing 0.05% of ProClin300. The sticks were incubated in 250 μl of cow plasma containing 0 ng/ml of LH in order to compare the effect of the various percentages of normal rabbit serum on the nonspecific signal, i.e. the colored background noise observed on the stick in the absence of any LH in the plasma. In this example, the quantification of the colored signal is carried out as described in Example 2 above. FIG. 3 is a photograph of the sticks after revealing.

TABLE 3
Strength of the coloration obtained according to the presence or
absence of normal rabbit serum in the conjugate solution

| | Without | | | | | |
|---|---|---|---|---|---|---|
| | serum | +5% | +10% | +20% | +25% | +50% |
| LH (ng/ml) | 0 | 0 | 0 | 0 | 0 | 0 |
| Quantification | 16.66 | 9.89 | 10.08 | 8.61 | 10.12 | 9.08 |
| of the colored | | | | | | |
| signal | | | | | | |

The addition of 5% to 50% of normal rabbit serum to the AC2 HRP decreases the background noise on the stick and makes it possible to improve the sensitivity of the test compared with a conjugate solution not containing in normal rabbit serum. In other words, the implementation of a method for detecting the spike in LH using a composition of conjugates according to the invention decreases the nonspecific signal. The concentration of 5% was retained for carrying out Examples 5 to 8.

### Example 5

### Comparative Tests of the Addition of 5% to 25% of Normal Rabbit Serum to the AC2 HRP Prepared at 10 μg/ml

The AC2 HRP was prepared at 10 μg/ml according to the method described in Example 1 above, in 5% or 25% by volume of normal rabbit serum, then dissolved in PBS-FCS 50% containing 0.05% of ProClin300. The sticks were incubated in 250 μl of cow plasma containing 0 ng/ml or 5.8 ng/ml of LH, then in the AC2 HRP and, finally, in the TMB membrane, in order to compare the effect of the various percentages of normal rabbit serum on the background noise and the signal obtained on the sticks. In this example, the quantification of the colored signal was carried out as described in Example 2 above. FIG. 4 is a photograph of the sticks after revealing.

TABLE 4
Strength of the coloration obtained according to the presence or
absence of normal rabbit serum in the conjugate solution

| | Without serum | | +5% | | +25% | |
|---|---|---|---|---|---|---|
| LH (ng/ml) | 0 | 5.8 ng/ml | 0 | 5.8 ng/ml | 0 | 5.8 ng/ml |
| Quanti- | 24.25 | 44.82 | 7.3 | 33.35 | 10.28 | 39.13 |
| fication of | | | | | | |
| the colored | | | | | | |
| signal | | | | | | |

The increase in the percentage of normal rabbit serum used to prepare the AC2 HRP at 10 μg/ml does not modify the strength of the signal obtained on the stick with a plasma having a high concentration of LH, but reduces the nonspecific signal obtained with a plasma at 0 ng/ml. The preparation of the AC2 HRP in 5% or 25% of normal rabbit serum gives an equivalent result.

In other words, the implementation of a method for detecting the spike in LH using a buffer solution of conjugate comprising an anti-LH antibody coupled to an enzyme, 50% of fetal calf serum and 5% or 25% of normal rabbit serum increases the sensitivity of detection.

### Example 6

### Comparative Tests of the Addition of Normal Rabbit Serum and of Glycerol to the AC2 HRP Prepared at 5 μg/ml

The AC2 HRP was prepared at 5 μg/ml according to the method described in Example 1 above, in 5% by volume of normal rabbit serum, then diluted in PBS-FCS 50% by volume containing 0.05% by volume of ProClin300 and also comprising from 1% to 50% by volume of glycerol (supplier Sigma, reference G7893). The sticks were incubated in 250 μl of cow plasma containing 0 ng/ml or 7 ng/ml of LH, then in the AC2 HRP and, finally, in the TMB membrane, in order to compare the effect of the different percentages of glycerol on the background noise and the signal present on the sticks. The quantification of the colored signal was carried out as described in Example 2 above. FIG. 5 is a photograph of the sticks after revealing.

TABLE 5
Strength of the coloration obtained as a function of the amount of
glycerol present in the conjugate solution

| | Without | | 1% | | 2% | | 3% | | 4% | | 5% | | 10% | |
|---|---|---|---|---|---|---|---|---|---|---|---|---|---|---|
| | glycérol | | glycerol | | glycerol | | glycerol | | glycerol | | glycerol | | glycerol | |
| LH (ng/ml) | 0 | 7 | 0 | 7 | 0 | 7 | 0 | 7 | 0 | 7 | 0 | 7 | 0 | 7 |
| Quantification | 10.28 | 26.38 | 7.77 | 24.31 | 9.60 | 21.95 | 9.60 | 21.99 | 4.75 | 24.06 | 4.47 | 18.49 | 5.13 | 17.67 |
| of the colored | | | | | | | | | | | | | | |
| signal | | | | | | | | | | | | | | |

| | 20% | | 30% | | 40% | | 50% | |
|---|---|---|---|---|---|---|---|---|
| | glycerol | | glycerol | | glycerol | | glycerol | |
| LH (ng/ml) | 0 | 7 | 0 | 7 | 0 | 7 | 0 | 7 |
| Quantification | 1.08 | 7.89 | 1.03 | 3.84 | 1.18 | 0 | 0.66 | 0.38 |
| of the colored | | | | | | | | |
| signal | | | | | | | | |

At 5 μg/ml of AC2 HRP, the addition of 1% to 4% of glycerol decreases the background noise, without decreasing the signal. Above 5%, the glycerol reduces not only the background noise, but also the specific signal. In other words, the implementation of a method for detecting the spike in LH using a buffer solution of conjugate comprising an anti-LH antibody coupled to an enzyme, 50% by volume of fetal calf serum, 5% of normal rabbit serum and from 1% to 4% of glycerol decreases the background noise without modifying the detection of the spike in LH.

### Example 7

### Comparative Tests of the Addition of Normal Rabbit Serum and of Glycerol to the AC2 HRP Prepared at 10 μg/ml

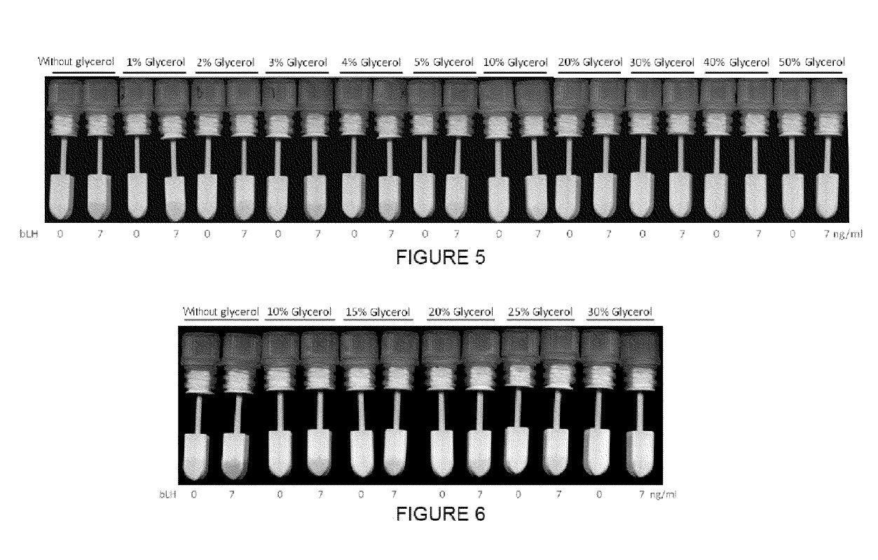

The AC2 HRP was prepared at 10 μg/ml according to the method described in Example 1 above, in 5% by volume of normal rabbit serum, and supplemented with a solution of PBS-FCS 50% by volume containing 0.05% by volume of ProClin300 and 10% to 50% by volume of glycerol (supplier Sigma, reference G7893). The sticks were incubated in 250 μl of cow plasma containing 0 ng/ml or 7 ng/ml of LH, then in the AC2 HRP and, finally, in the TMB membrane, in order to compare the effect of the various percentages of glycerol on the background noise and the signal present in the sticks. In this example, the quantification of the colored signal was carried out as described in Example 2 above. FIG. 6 is a photograph of the sticks after revealing.

TABLE 6
Strength of the coloration obtained according to the amount of
glycerol present in the conjugate solution

| | Without | | 10% | | 15% | | 20% | | 25% | | 30% | |
|---|---|---|---|---|---|---|---|---|---|---|---|---|
| | glycerol | | glycerol | | glycerol | | glycerol | | glycerol | | glycerol | |
| LH (ng/ml) | 0 | 7 | 0 | 7 | 0 | 7 | 0 | 7 | 0 | 7 | 0 | 7 |
| Quantification | 14.5 | 51.87 | 6.68 | 30.6 | 6.52 | 28.81 | 4.95 | 17.95 | 4.06 | 15.67 | 2.28 | 10.85 |
| of the colored | | | | | | | | | | | | |
| signal | | | | | | | | | | | | |

At 10 μg/ml of AC2 HRP, the addition of 10% or 15% of glycerol decreases the background noise, while at the same time reducing the signal. Above 15% by volume of glycerol, the quenching of the specific signal is too great, making the visual detection of a spike in LH impossible. The best sensitivity of the test is obtained with the AC2 HRP prepared at 10 μg/ml in 5% of normal rabbit serum and 10% or 15% by volume of glycerol. In other words, the implementation of a method for detecting the spike in LH using a buffer solution of conjugate comprising an anti-LH antibody coupled to an enzyme and 50% by volume of fetal calf serum and 5% by volume of normal rabbit serum and 10% or 15% of glycerol increases the sensitivity of detection.

### Example 8

### Comparative Tests in the Case of Storage at 4° C. or −20° C. of the AC2 HRP Prepared at 5 μg/ml

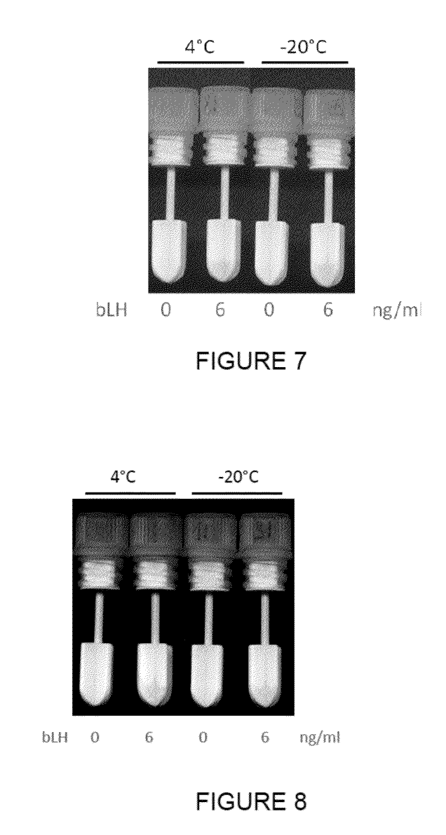

The AC2 HRP was prepared at 5 μg/ml according to the method described in Example 1 above, in 5% by volume of normal rabbit serum, and supplemented with a solution of PBS-FCS 50% by volume containing 0.05% by volume of ProClin300 and 1% by volume of glycerol (supplier Sigma, reference G7893), and stored at 4° C. or at −20° C. for 3 weeks. After 3 weeks, the sticks were incubated in 250 μl of cow plasma at 0 ng/ml or 7 ng/ml of LH, then in the AC2 HRP stored at 4° C. or at −20° C., and, finally, in the TMB membrane, in order to compare the storage of the AC2 HRP at 4° C. and at −20° C. In this example, the quantification of the colored signal was carried out as described in Example 2. FIG. 7 is a photograph of the sticks after revealing.

TABLE 7
Strength of the coloration obtained according
to the conjugate solution storage temperature

| | |
|---|---|
| 4° C. | −20° C. |

| LH (ng/ml) | 0 | 6 | 0 | 6 |
|---|---|---|---|---|
| Quantification of | 5.35 | 27.75 | 3.27 | 28.72 |
| the colored signal | | | | |

The AC2 HRP prepared at 5 μg/ml in 5% by volume of normal rabbit serum and 1% by volume of glycerol and stored at 4° C. or at −20° C. exhibits the same performance levels: the same color strength is obtained on the sticks. This example therefore clearly demonstrates that the composition according to the invention advantageously makes it possible to store a composition of conjugates at between −20 and 4° C. In addition, the composition according to the invention advantageously makes it possible to preserve the biological activity of the antibodies/enzymes included in the composition.

### Example 9

### Comparative Tests in the Case of Storage at 4° C. or −20° C. of the AC2 HRP Prepared at 10 μg/ml

The AC2 HRP was prepared at 10 μg/ml according to the method described in Example 1 above in 5% by volume of normal rabbit serum, and supplemented with a solution PBS-FCS 50% by volume containing 0.05% by volume of ProClin300 and 10% by volume of glycerol (supplier Sigma, reference G7893), and stored at 4° C. or at −20° C. for 3 weeks. After 3 weeks, the sticks were incubated in 250 μl of cow plasma containing 0 ng/ml or 7 ng/ml of LH, then in the AC2 HRP stored at 4° C. or at −20° C., and, finally, in the TMB membrane, in order to compare the storage of the AC2 HRP at 4° C. and at −20° C. In this example, the quantification of the colored signal was carried out as described in Example 2 above. FIG. 8 is a photograph of the sticks after revealing.

TABLE 8
Strength of the coloration obtained according to
the storage temperature of the conjugate solution

| | |
|---|---|
| 4° C. | −20° C. |

| LH (ng/ml) | 0 | 6 | 0 | 6 |
|---|---|---|---|---|
| Quantification of | 3.27 | 20.92 | 2.6 | 20.58 |
| the colored signal | | | | |

The AC2 HRP prepared at 10 μg/ml in 5% by volume of normal rabbit serum and 1% by volume of glycerol and stored at 4° C. or at −20° C. exhibits the same performance levels: the same color strength is obtained on the sticks.

As demonstrated in this example, the medium for preparing the AC2 HRP thus makes it possible to store the conjugated antibody at 4° C. as effectively as storage at −20° C.

### Example 10

### Performance Levels of a Kit for Detecting the Pre-ovulatory Spike in LH, Stored at 4° C. with the AC2 HRP Prepared in the Medium Described and Claimed

The effect of the medium was tested in the case where all the elements of the kit produced according to the method below were stored at 4° C. for 10 weeks. In this example, the kit comprises a stick comprising test surfaces, ready to use in a heparinized tube, a tube comprising the conjugate solution, namely a buffer solution with an anti-LH antibody coupled to horseradish peroxidase (conjugated antibody), and a tube containing a solution comprising the substrate for horseradish peroxidase, namely the TMB membrane called substrate. The material used was immunosticks sold by the company Nunc, and three screw-cap cryotubes sold by the company Nunc.

A heparinized tube was prepared according to the method described in Example 2.

The sticks were prepared by coating with 250 μl of rabbit anti-bovine LH antibody purified on a protein A sepharose column from an immune serum produced in rabbits by the provider Eurogentec (Belgium) according to the “Standard anti-protein packages 28-day Speedy in Rabbit” (trademark) method, said antibody being referred to below as AC1, prepared at 20 μg/ml in 0.1M NaHCO₃/Na₂CO₃ buffer, pH 9.6, containing 0.05% by volume of ProClin300. In another embodiment, the anti-LH antibody (AC1) was a rabbit anti-porcine LH antibody; it is the antibody produced, for example, by immunization of rabbits according to the method described, for example, in the literature reference Pelletier J., and al., 1968 [5]. This involved the method comprising, for example, three injections of 200 μg of purified porcine LH sold by the company Tucker Endocrine Research Institute LLC TUENRE (Atlanta, USA), which were carried out every two weeks, followed by ten boosters with 100 μg of purified porcine LH performed every five weeks. The bleeds were carried out six and nine days after each booster and 40 ml of blood were recovered. The rabbit antibodies were purified from the blood samples by affinity chromatography on protein A sepharose gel according to the method described in “Techniques Immuno-enzymatiques” [“Immunoenzymatic Techniques”], Thérèse Ternynck et al., INSERM publications, 1987 [1]. The stick was coated with the antibody according to the method described in Thérèse Ternynck et al. 1987 [1]: incubation for one hour at 37° C., then for 18 hours at 4° C. Draining of the stick by shaking. Saturation coating of the stick in 900 μl of PBS supplemented with 50% fetal calf serum (FCS), for 1 hour at 37° C. Draining of the stick by shaking. Drying of the stick vertically in an incubator at 37° C. for 3 hours. Storage of the stick at 4° C. in a dried heparinized tube.

The horse anti-ovine LH antibody coupled to horseradish peroxidase (HRP), referred to below as AC2 HRP, was prepared at 5 or 10 μg/ml in PBS-FCS 50% by volume containing 0.05% by volume of ProClin300, with incubation for 50 minutes at 37° C. Three conditions were studied: a) AC2 HRP prepared at 5 μg/ml in 5% of normal rabbit serum and 1% of glycerol, b) AC2 HRP prepared at 5 μg/ml in 5% of normal rabbit serum and 3% of glycerol, and c) AC2 HRP prepared at 10 μg/ml in 5% of normal rabbit serum and 15% of glycerol.

The AC2 HRP antibody was produced as described in Example 2. In another embodiment, it is the antibody produced, for example, by immunization of rabbits according to the method described in the literature reference Pelletier J. et al., 1968 [5]. This involved the method comprising, for example, three injections of 200 μg of purified porcine LH originating from the company Tucker Endocrine Research Institute LLC TUENRE (Atlanta, USA), which were carried out every two weeks, followed by ten boosters with 100 μg of purified LH, performed every five weeks. The bleeds were carried out six and nine days after each booster and 40 ml of blood were recovered. The rabbit antibodies were purified from the blood samples by affinity chromatography on protein A sepharose gel according to the method described in Thérèse Ternynck et al., 1987 [1]. The coupling of the antibody was carried out according to the method described in Thérèse Ternynck et al., 1987 [1]. The porcine LH was sold by Tucker Endocrine Research Institute LLC TUENRE (Atanta, USA). The peroxidase used is sold by Sigma under the reference P6782. The revealing agent tubes (tube No. 3) were prepared by distributing 300 μl of TMB membrane per cryotube. In order to evaluate the effectiveness of the medium, the performance levels of the kits stored for 10 weeks at 4° C. were compared with those obtained just after the production of the kits, before any storage at 4° C. In this example, the quantification of the colored signal was carried out as described in Example 2 above. The results are given in Table 9 below:

TABLE 9
Strength of the coloration obtained according to the storage or non-
storage of the kit for implementing the method according to the invention

| | AC2 5 μg/ml in | | AC2 5 μg/ml in | | AC2 10 μg/ml in | |
|---|---|---|---|---|---|---|
| | 5% rabbit serum and | | 5% rabbit serum and | | 5% rabbit serum and | |
| | 1% glycerol | | 3% glycerol | | 15% glycerol | |
| Quantification | At | Storage for | At | Storage for | At | Storage for |
| of the stick | production | 10 weeks | production | 10 weeks | production | 10 weeks at |
| color | output | at 4° C. | output | at 4° C. | output | 4° C. |
| LH 0 ng/ml | 10.83 | 7.18 | 11.32 | 6.61 | 9 | 9.17 |
| LH 6 ng/ml | 30.76 | 30.71 | 24.36 | 21.03 | 19.17 | 19.74 |

It was observed that, under the three AC2 HRP preparation conditions, the strength of the colored signal of the stick, obtained with a plasma at 0 ng/ml of LH or with a plasma at 6 ng/ml of LH, is not statistically different with a kit at production output or stored at 4° C. for 10 weeks. These results therefore clearly demonstrate that the composition of said medium for preparing the AC2 HRP allows storage of the kit at 4° C. without impairment of the performance levels of the test from the point of view of its sensitivity and its stability.

The example was also carried out with a composition comprising the AC2 HRP at 20 μg/ml, prepared in PBS-FCS 50% containing 5% of normal rabbit serum and 10% of glycerol. The quantification of the colored signal obtained was carried out on sticks at production output and after 5 months of storage at 4° C.

TABLE 10
Strength of the coloration obtained according to
the storage or non-storage of the kit for implementing
the method according to the invention

| Quantification of | AC2 20 μg/ml in 5% rabbit serum and 10% glycerol | |
|---|---|---|
| the stick color | At production output | Storage for 5 months at 4° C. |
| LH 0 ng/ml | 12.6 | 1.7 |
| LH 6 ng/ml | 39.24 | 37.95 |

It was observed that the strength of the colored signal of the stick, obtained with a plasma at 0 ng/ml of LH or with a plasma at 6 ng/ml of LH, is not statistically different between a kit at production output and a kit stored at 4° C. for 5 months. These results therefore clearly demonstrate that the composition of said medium for preparing the AC2 HRP allows storage of the kit at 4° C. without impairment of the performance levels of the test from the point of view of both its sensitivity and its stability.

### LIST OF REFERENCES

1. “Techniques Immuno-enzymatiques” [“Immunoenzymatic Techniques”], Thérèse Ternynck and Stratis Avrameas, INSERM publications, 1987,

2. Immunobiology, Charles A. Janeway, Paul Travers, Pierre L. Masson—2003—Medical; Janeway's Immunobiology, Kenneth Murphy, Paul Travers, Mark Walport, 2011, GS publications

3. “Enzyme-linked immunosorbent assay (ELISA). Quantitative assay of immunoglobulin G”, Engvall, E. and Perlman, P., Immunochemistry, 1971 September; 8(9):871-4 PMID: 5135623

4. “Enzyme-Linked Immunosorbent Assay”, Goldsby, R. A., Kindt, T. J., Osborne, B. A. and Kuby, J., in Immunology, 5th edition (2003), pp. 148-150., W. H. Freeman, New York.

5. “Dosage radio-immunologique de I'hormone lutéinisante plasmatique chez le mouton. Mise au point de la technique de dosage” [“Radioimmunological assay of plasma luteinizing hormone in sheep. Development of the assay technique”], Pelletier J., Kann G., Dolais J., Rosselin G., C. R. Acad. Sc. Paris, 1968 June; 266: 2291-2294

1. A composition of conjugates comprising an antibody coupled to an enzyme, at least two different mammalian sera, and glycerol.

2. The composition as claimed in claim 1, wherein one of said at least two sera is normal mammalian serum.

3. The composition as claimed in claim 2, wherein said normal mammalian serum is normal human or animal serum.

4. The composition as claimed in claim 3, wherein said normal animal serum is selected from the group comprising normal serum from a farm animal or from a pet.

5. The composition as claimed in claim 4, wherein the normal serum from a farm animal or from a pet is selected from the group comprising bovines, pigs, members of the ovine family, members of the goat family, members of the camel family, members of the horse family, murines, canines, felines and primates.

6. The composition as claimed in claim 2, wherein the normal mammalian serum is normal rabbit serum.

7. The composition as claimed in claim 1, wherein one of said at least two sera is human or animal mammalian serum.

8. The composition as claimed in claim 7, wherein the animal serum is selected from the group comprising serum from a farm animal or from a pet.
